(19) United States
(12) Patent Application Publication
(10) Pub. No.: US 2024/0122817 A1
(43) Pub. Date: Apr. 18, 2024

(54) FACE CONTOUR MASK SYSTEM AND METHOD

(71) Applicant: Matthew T. Thompson, Shingle Springs, CA (US)

(72) Inventor: Matthew T. Thompson, Shingle Springs, CA (US)

(21) Appl. No.: 18/234,277

(22) Filed: Aug. 15, 2023

Related U.S. Application Data
(63) Continuation of application No. 18/084,440, filed on Dec. 19, 2022, now abandoned.
Application No. 18/084,440 is a continuation of application No. 17/991,705, filed on Nov. 21, 2022, now abandoned.
Application No. 17/991,705 is a continuation of application No. 16/865,281, filed on May 1, 2020, now abandoned.
Application No. 16/865,281 is a continuation of application No. 15/962,597, filed on Apr. 25, 2018, now abandoned.
(60) Provisional application No. 62/520,149, filed on Jun. 15, 2017.

(51) Int. Cl. A61K 8/02; A45D 40/30; A45D 44/00

(52) U.S. Cl. CPC A61K 8/0212; A45D 40/30; A45D 44/002; A45D 44/22

(57) ABSTRACT

Provided are embodiments of system and method for creating shadowed blend using a face contour mask system. The contour mask system may include a faceplate which can be adapted to cover a plurality of portions of a face of a user. The faceplate may include two elongated openings adapted to fit over the eyes of the user. An annular ridge, protruding outwardly from the back side of the faceplate, may encircle each elongated opening. The faceplate may include a spacer to allow a space gap between the face of the user and the faceplate when the faceplate is positioned or worn over the user's face.

### CROSS-REFERENCE TO RELATED APPLICATION

The present application is a continuation of U.S. patent application Ser. No. 18/084,440, filed Dec. 19, 2022, which is a continuation of U.S. patent application Ser. No. 17/991,705, filed Nov. 21, 2022, now abandoned, which is a continuation of U.S. patent application Ser. No. 16/865,281, filed May 1, 2020, now abandoned, which is a continuation of U.S. patent application Ser. No. 15/962,597, filed Apr. 25, 2018, now abandoned, which claims priority to U.S. Provisional Application No. 62/520,149, filed Jun. 15, 2017, which are hereby incorporated by reference in their entireties.

### TECHNICAL FIELD

The claimed invention relates to face contour mask system and method, and in some aspects to a face contour mask system and method for creating shadowed blend.

### BACKGROUND

Creating makeup or tanning contour has been a popular trend in the beauty world. In countoring, the user often wants to shade areas or regions of her face to define or reshape, for example, the forehead, chin, and cheekbones, with shade that is darker than ther skin color. Current contouring products and methods include using contouring airbrush and sunless contouring solutions. A problem with such processes is that the placement of the darkened areas or regions is typically up to the wearer who is most often, not a qualified make-up artist with the knowledge of where and how to apply contouring solutions. These products take much time to apply, are uneven and often create an unnatural “caked-on” artificial look. In addition, these solutions only last for a short time and are easily washed off with a single bathing.

Accordingly, there is a need for a system and method that simplifies the process of creating makeup or tanning contour.

### SUMMARY

Provided herein are embodiments of system and method for creating shadowed blend using a face contour mask system. In some embodiments, the contour mask system may include a faceplate which can be adapted to cover a plurality of portions (or regions) of a face of a user, thus leving other areas of exposed skin. The faceplate may include two elongated apertures adapted to fit over the eyes of the user. On the inside or back side of the faceplate, an annular ridge may encircle each elongated aperture. The ridges protrude outwardly from the back side of the faceplate. The faceplate may include a flexible nose rest positioned at the foot of each ridge. The flexible nose rest may be reinforced with bendable metal wire. The ridges and the nose rests may allow a space gap between the face of the user and the faceplate when the faceplate is positioned or worn over the user's face. Because the nose rest is flexible, the user may adjust the nose rest to increase or decrease the space gap, or distance between the face of the user and the faceplate.

In some embodiments, the faceplate may include rounded edges, and removable ultraviolet ray protective eyewear.

In some operations, the user may start placing the faceplate to fit over the face of the user. The user then may bend the flexible nose rests to adapt the nose rests to fit over the nose bridge of the user when placing the back side of the faceplate over the face of the user, so that the nose rests may create a space gap between the face of the user and the back side of the faceplate. Once the faceplate is positioned over the face of the user with a desired space gap and desired areas of exposed skin, the user may apply a sprayed solution or tanning rays over desired areas of exposed skin. With the provided space gap, the applied substance or tan may be blurred instantly to all correct/desired areas while being properly blended at the same time, as to advantageously avoid any hard lines.

Other features and advantages of the present invention will be or will become apparent to one with skill in the art upon examination of the following figures and detailed description, which illustrate, by way of examples, the principles of the present invention.

### BRIEF DESCRIPTION OF THE DRAWINGS

The present invention may be better understood by referring to the following figures. The components in the figures are not necessarily to scale, emphasis instead being placed upon illustrating the principles of the disclosure. In the figures, like reference numerals designate corresponding parts throughout the different views.

FIG. 1 illustrates an exemplary front view of a face mask contour system, according to some aspects of the disclosure.

FIG. 2 illustrates an exemplary side view of the face mask contour system of FIG. 1, according to some aspects of the disclosure.

FIG. 3 illustrates an exemplary back view of the face mask contour system of FIG. 1, according to some aspects of the disclosure.

FIG. 4 illustrates an exemplary close-up view of a section of the back view of the face mask contour system of FIG. 1, according to some aspects of the disclosure.

FIG. 5 illustrates an exemplary process for tanning or makeup application, using the face mask contour system, according to some aspects of the disclosure.

FIG. 6 illustrates an exemplary front view of another faceplate of the face mask contour system, according to some aspects of the disclosure.

FIG. 7 illustrates an exemplary perspective view of another face mask contour system, according to some aspects of the disclosure.

FIG. 8 illustrates an exemplary side view of the face mask contour system of FIG. 7, according to some aspects of the disclosure.

FIG. 9 illustrates an exemplary back view of the face mask contour system of FIG. 7, including a bit mount and an extension arm, according to some aspects of the disclosure.

FIGS. 10A-10B illustrate exemplary perspective views of the face mask contour system of FIG. 7, including a head mount, according to some aspects of the disclosure.

FIG. 11A illustrates exemplary front view and exemplary side view of a user's face prior to using a face mask contour system, according to some aspects of the disclosure.

FIG. 11B illustrates exemplary front view and exemplary side view of a user's face of FIG. 11A during use of a face mask contour system, according to some aspects of the disclosure.

FIG. 11C illustrates exemplary front view and exemplary side view of a user's face of FIG. 11A after use of a face mask contour system of FIG. 11B, according to some aspects of the disclosure.

FIG. 12A illustrates six exemplary front views of various face and head shapes prior to using a face mask contour system, according to some aspects of the disclosure.

FIG. 12B illustrates six exemplary front views of various face and head shapes of FIG. 12A during use of a face mask contour system, according to some aspects of the disclosure.

FIG. 12C illustrates six exemplary front views of various face and head shapes of FIG. 12A after use of a face mask contour system of FIG. 12B, according to some aspects of the disclosure.

FIG. 13A illustrates five exemplary front views of various face and head shapes of five different users prior to using a face mask contour system, according to some aspects of the disclosure.

FIG. 13B illustrates five exemplary front views of various face and head shapes of five different users of FIG. 13A during the uses of a face mask contour system, using five different faceplates having different shapes and/or sizes, according to some aspects of the disclosure.

FIG. 13C illustrates five exemplary front views of various face and head shapes of five different users of FIG. 13A after the uses of a face mask contour system, using five different faceplates having different shapes and/or sizes of FIG. 13B, according to some aspects of the disclosure.

### DETAILED DESCRIPTION

The below described figures illustrate the described invention and method of use in at least one of its preferred, best mode embodiment, which is further defined in detail in the following description. Those having ordinary skill in the art may be able to make alterations and modifications to what is described herein without departing from its spirit and scope. While this invention is susceptible of embodiment in many different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

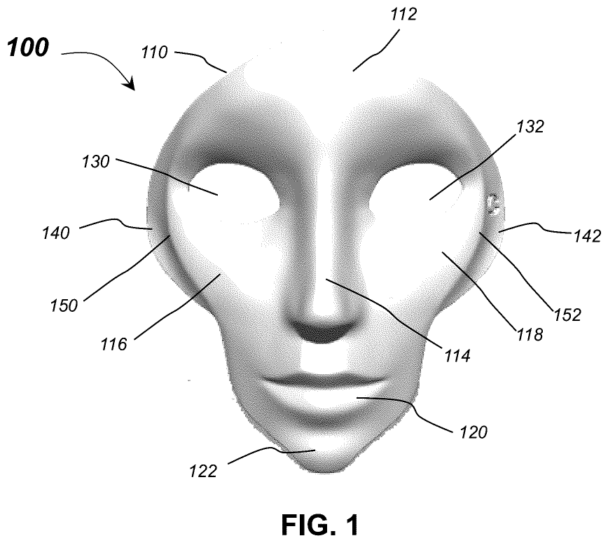

FIGS. 1 to 13 illustrate exemplary embodiments of system and method for creating shadowed blend using a face contour mask system. In some embodiments, the contour mask system generally may include a faceplate adapted to be positioned over the face of a user to cover a plurality of regions of the user's face. The faceplate may be made of lightweight, durable and water-resistant material. It may be stowed, compact, and comfortable to wear. The faceplate may include a front side and a back side. When the faceplate is positioned over the user's face, the front side faces away from the user's face, and the back side faces toward the user's face. Generally, the faceplate, when positioned over the user's face, may cover only certain portions (or regions) of the user's face for example, forehead, face, eyes, cheeks, chin, jawline, mouth and nose regions. The covered regions may prevent tanning or contouring solutions from being applied to the user's face during application, while allowing the desired application areas of the face to remain exposed during application to receive tanning or receive contouring solutions for producing a semi-permanent facial “contour”, either artificially or naturally (e.g., via ultraviolet (UV) tanning) to create an artificially shadowed effect of a more dramatic bone structure on the user's face, and for creating the illusion of symmetry, making the user more subjectively beautiful.

In some embodiments, the faceplate may include two elongated apertures (or openings) over the eye portions (or regions) of the eyes. In certain embodiments, the eye areas are made of clear UV protective lenses so that they can prevent application to the eyes while allowing vision and protection from ocular UV exposure. In other certain embodiments, the system may include a removable ultraviolet ray protective eyewear to cover the two elongated apertures to protect the eyes, for example, during a UV tanning process.

In various embodiments, the face mask contour system may allow the faceplate, when positioned over the face of a user, to provide an even space between the face of the user and the faceplate in some or all regions of the face. Such spacing may allow blurring or blending at and adjacent to the edges of the faceplate when applying sunless tan, sprayed or powdered dye, pigment, UV rays, makeup, and the like. The applied substances or rays may be blurred instantly to all correct/desired regions while being properly blended at the same time, as to advantageously avoid any hard lines. Hard lines often suggest an artificial application. Thus, without hard lines, the application may appear more natural and more subjectively beautiful.

The embodiments of the face mask contour system may be used with, but not limited to, UV tanning, sunless tanning solutions, airbrush/make-up contouring, and all other forms of beauty related products.

Turning to FIG. 1, an exemplary front view of a face mask contour system 100 is illustrated, according to some embodiments. The face mask contour system 100 may include a faceplate 110, the front side of which is shown in FIG. 1. In some embodiments, the faceplate 110 may emulate a perfect bone structure for nearly all people. The faceplate 110 may include forehead portion 112, nose portion 114, right upper cheek portion 116, left upper cheek portion 118, mouth portion 120, and chin portion 122. The faceplate 110 may also include right flange 140 and left flange 142. The flange 140 and 142 are connected to the cheek portions 116, 118 and forehead portion 112 at ridges 150 and 152 respectively. The faceplate 110 may include two elongated apertures (or openings) 130 and 132 over the eye portions.

When the faceplate 110 is positioned over the face of a user as described below, it leaves portions or regions of the face exposed to application such as sunless tanning solutions, makeup, UV rays to create a design of a desired contour.

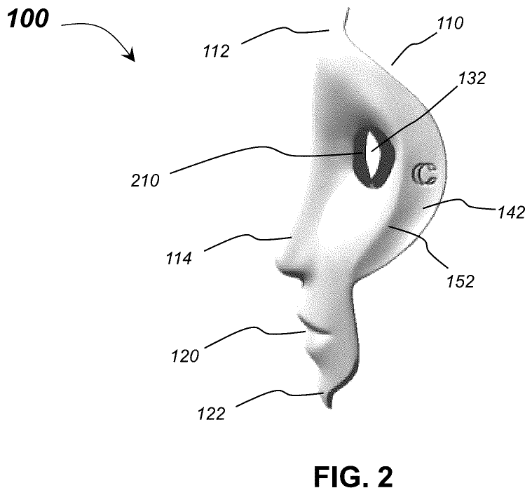

Turning to FIG. 2, an exemplary side view of the faceplate 110 is illustrated, according to some embodiments. In some embodiments, the faceplate 110 may include UV protective lens or goggle 210 as shown for aperture 132, and a corresponding lens or goggle (not shown) for the corresponding aperture 130 (not shown). The UV protective lenses may be made of clear, FDA approved UV protective lenses or goggles so that they can prevent application to the eyes while allowing vision and protection from ocular UV exposure.

As the faceplate 110 may be made of UV protective material, together with the UV protective lenses or goggles, the face mask contour system 100 may provide an all-in-one system for tanning to protect both the user's eyes and face from UV related damage, while achieving a natural contour of the user's own color.

The flange 142 and flange 140 (not shown) may curve steeper starting at ridge 152 and 150 (not shown). The steeper curves may allow the faceplate 110 to hug the sides of the face of the user closer. But note that, as described herein, the flange 140, 142 may not directly touch the face of the user. In some embodiments, the flange 140 and 142 may be bendable.

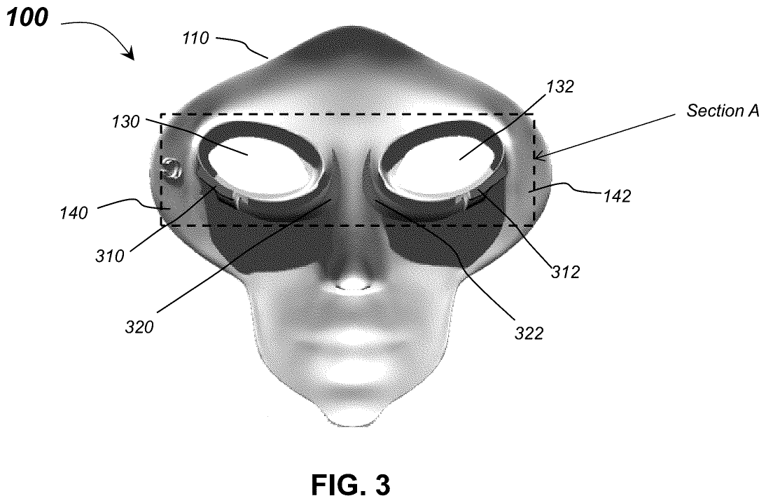

Turning to FIG. 3, an exemplary back view of the faceplate 110 is illustrated, according to some embodiments. The back view shows the back side of the faceplate 110. The faceplate 110 may include an annular ridge 310 encircling the elongated aperture 130 and protruding outwardly from the back side of the faceplate 110. Similarly, an annular ridge 312 encircling the elongated aperture 132 and protruding outwardly from the back side of the faceplate 110.

The faceplate 110 may also include a spacer to create a space gap between the user's face and the faceplate 110. In some embodiments, the spacer may be a flexible nose rest 320 fixedly coupled to the elongated aperture 310, and similarly a flexible nose rest 322 fixedly coupled to the elongated aperture 312. In some embodiments, the nose rests 320, 322 may include rubber housing with treaded feet, encapsulating metal wire for customizable flexibility to fit all nose types. In some exemplary operations, a user may bend the nose rest(s) into a position that rests comfortably on the user's nose while allowing the faceplate 110 to continue to maintain a distance from the face of the user. As described herein, the space may advantageously allow the user to create a shadowed blend when applying tanning or makeup solutions.

It is noted that although embodiments described herein include nose rests to advantageously create a space gap between the user's face and the face mask, other suitable means may also be used to create similar space gap. These may include, but not limited to, fixed or removable spacer, such as rubber spacers placed on the back side of the faceplate, head mount, elastic band or string, clear plastic band, mouth bite mount, and silicone skin adhesive.

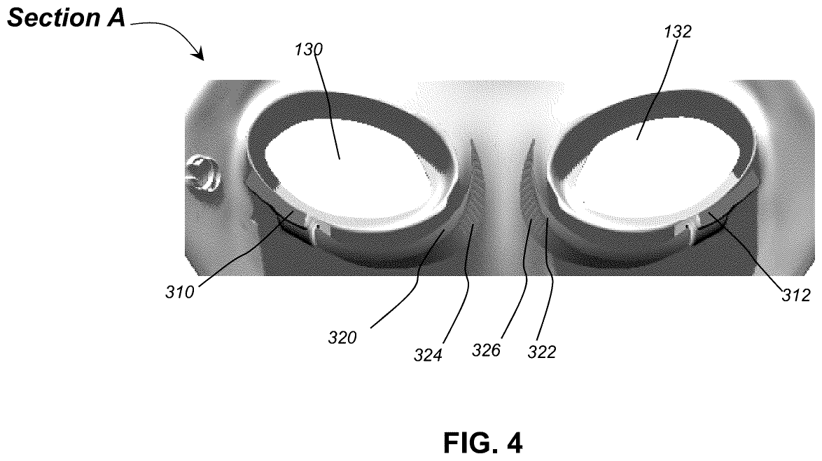

FIG. 4 shows an exemplary close-up view of the Section A of the back view of the faceplate 110 as shown in FIG. 3. As described above, the nose rests 320, 322 may include rubber housing with treaded feet 324, 326 respectively, encapsulating metal wire for customizable flexibility to fit all nose types.

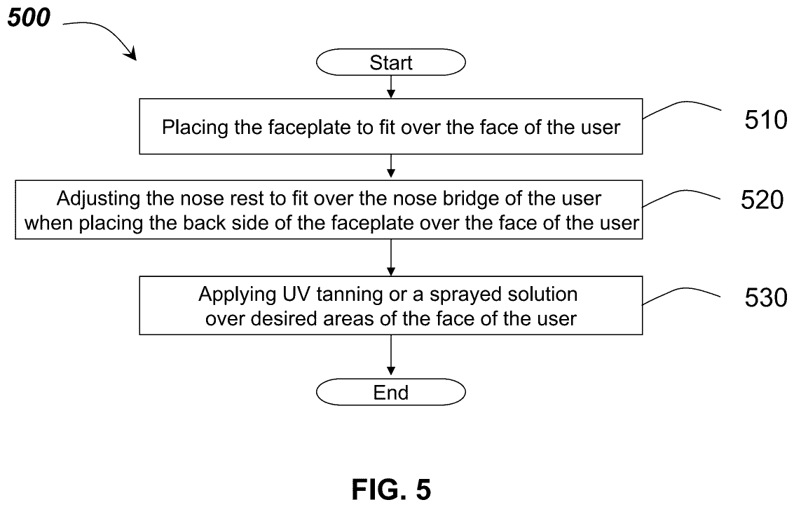

Turning to FIG. 5, an exemplary process 500 for UV tanning or makeup application, using the face mask contour system 100 is illustrated, according to some embodiments. At 510, the user may start the process with placing the faceplate 110 to fit over the face of the user. At this time, the faceplate 110 may cover the desired portions of the user's face, for example, the forehead, upper cheeks, mouth, and chin portions. At 520, the user may adjust the flexible nose rests 320 and 322 to adapt the nose rests to fit over the nose bridge of the user's nose when placing the back side of the faceplate 110 over the face of the user. The nose rests may create a space gap between the face of the user and the back side of the faceplate 110. The user may repeat step 520 until the faceplate 110 is positioned over all the desired portions of the face, and a desired space gap is achieved.

At 520, the faceplate 110 may include clear UV protective lenses over the eye portions to prevent application to the eyes. In other certain embodiments, where the faceplate 110 may not include clear UV protective lenses, the user may attach removable ultraviolet ray protective eyewear to cover the two elongated apertures 130, 132 before positioning the faceplate 110 over the user's face.

At 530, the user may apply a sprayed solution over desired portions or regions of the face of the user. These regions are generally around the faceplate 110. With the provided space gap in step 520, the applied substance may be blurred instantly to all correct/desired regions while being properly blended at the same time, as to advantageously avoid any hard lines.

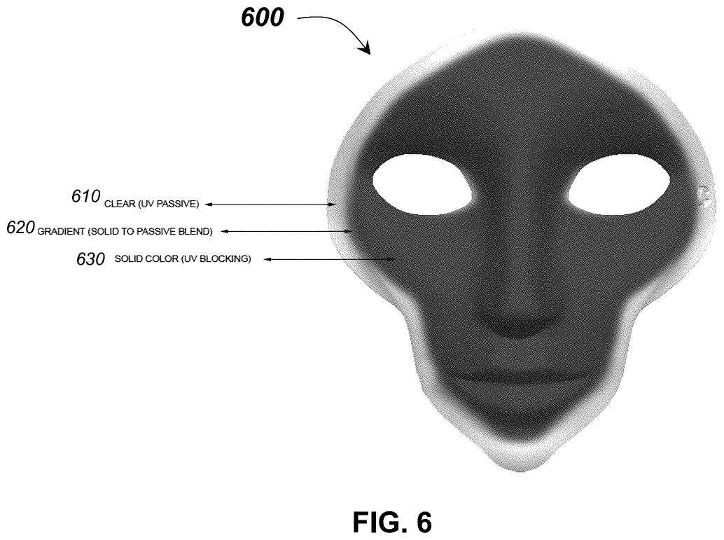

Turning to FIG. 6, an exemplary front view of a faceplate 600 is illustrated, according to some embodiments. In some embodiments, the faceplate 600 may include many parts similar to those described above for the faceplate 110 in FIGS. 1-4. However, the faceplate may not include a nose rest. As a result, the faceplate 600 may rest directly on the face of the user when used, not requiring a space gap. In addition, in some embodiments, the faceplate 600 may be specially made where the faceplate 600 may be clear around the outer edges with a gradient blending into a solid color toward the center of the faceplate. This design may advantageously lend itself to customizability for multiple face-types. The faceplate 600 may be suitable for UV tanning application, but not for sunless spray tanning, to achieve contour, blending results.

FIG. 6 shows a clear (UV passive) area 610 encircling the outer most edge of the faceplate 600. Adjacent to the area 610, and toward the center of the faceplate 600, is gradient (solid to passive blend) area 620 encircling the faceplate 600. For the remaining area 630 of the faceplate 600, it may be made of solid color with UV blocking capability.

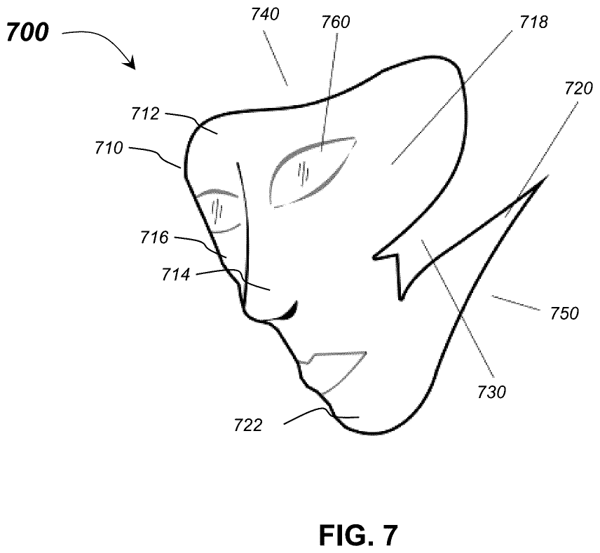

Turning to FIG. 7, an exemplary perspective view of a face mask contour system 700 is illustrated, according to some embodiments. The face mask contour system 700 may include a faceplate 710, the perspective view of which is shown in FIG. 7. Similar to the faceplate 110, the faceplate 710 may include a plurality of portions or regions, e.g., forehead portion 712, nose portion 714, right upper cheek portion 716, left upper cheek portion 718, left lower cheek portion 720, right lower cheek portion (not shown), and chin portion 722. The eye portion 760 may be covered with eyecup having UV blocking lens. When the faceplate 710 is positioned over the face of a user as described below, it leaves portions or regions of the face (e.g., cheek region 730, forehead region 740, and jawline region 750) exposed to application such as sunless tanning solutions, makeup, UV rays to create a design of a desired contour.

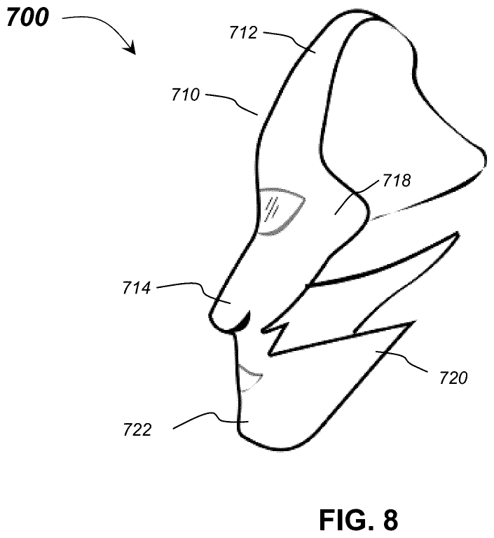

FIG. 8 illustrates an exemplary side view of the faceplate 710.

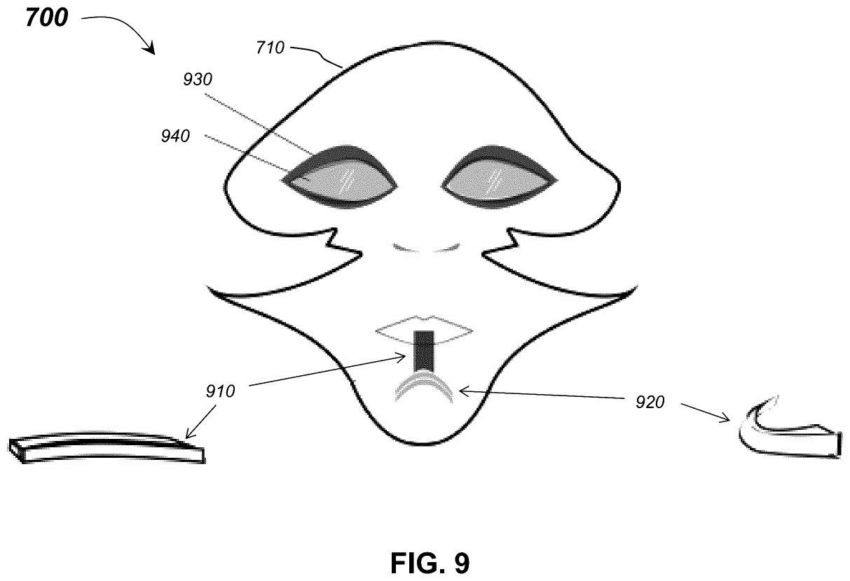

Turning to FIG. 9, an exemplary back view of the face mask contour system 700 is illustrated, according to some embodiments. In particular, FIG. 9 illustrates the back view of the faceplate 710, an extension arm 910 and a bit mount 920. In some exemplary operations, the extension arm 910 and the bit mount 920 may be used to allow the user to float the faceplate 710 in front of the user's face, by providing a space gap between the user's face and the faceplate 710. The user may attach the bit mount 920 to one end of the extension arm 910, hold (e.g., bite on) the bit mount 920 in the user's mouth, and attach the other end of the extension arm 910 to the back (inside face) of the faceplate 710 before positioning the faceplate 710 over the user's face. In some embodiments, the length of the extension arm 910 may be adjustable. In other embodiments, the extension arm 910 may come in different lengths. The space gap between the user's face and the faceplate 710 may prevent hard/harsh lines during the application of UV rays, sunless tanning spray, dyes, makeup, and the like, regardless of the variation of face shapes from person to person. The distance causes the solution or UV rays to be blurred and/or blended at the edges or the regions of exposed skin. The extension arm 910 may be made of flexible, yet rigid material.

As shown in FIG. 9, the face mask contour system 700 may include integrated UV protective eyewear, with eyecups 930 and UV blocking lens 940. The UV blocking lenses may provide UV protected vision, while undergoing the tanning/contouring process. The eyecups 930 may also help the user to determine and hold the correct distance and placement of the faceplate 710 to the user's face.

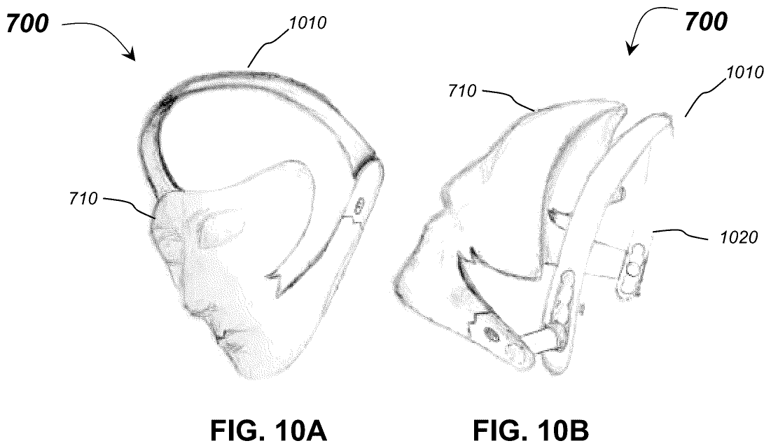

As illustrated in FIGS. 10A and 10B, the face mask contour system 700 may include head mount 1010 that may be removably attached to the faceplate 710 as shown. The head mount 1010 may also allow the user to float the faceplate 710 in front of the user's face at a distance so not to cause lines during the application of UV rays, sunless tanning spray, dyes, makeup, and the like. The distance may cause the solution or rays to be blurred or blended at the point of contact to the skin. The mounted arms 1020, which pass over the user's face, may be made of a clear composite to allow UV rays to freely pass through.

In some embodiments, the faceplate 110, 610, 710 may be made of vacuum pressed, molded Acrylonitrile Butadiene Styrene (ABS) plastic, machine grade aluminum, or a combination thereof. The faceplate 110, 610, 710 may be made of different sizes and/or different shapes. The faceplates 110, 610, 710 may include rounded edges. Under the laws of fluid dynamics, the rounded edges may cause sprayed or kinetically powered darkening solutions/particles in motion to roll over and dissipate into a more blurred/blended contour on the skin at the edge of the applied substance.

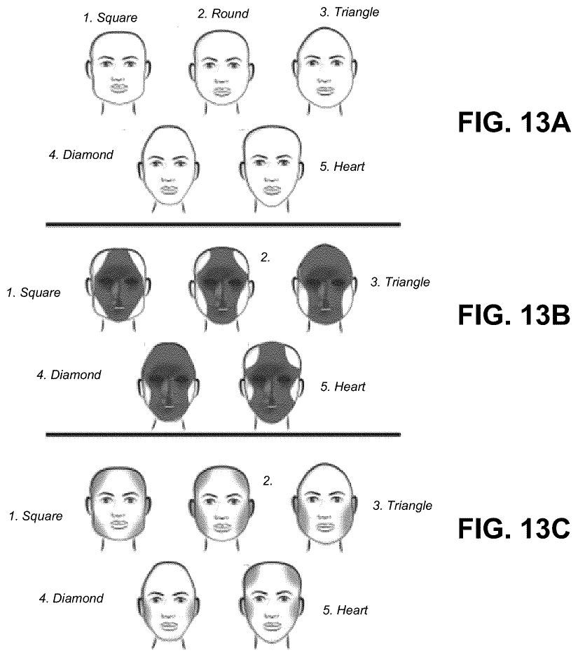

It should be noted that although the faceplate 110, 610, 710 are illustrated in the drawings with certain shapes, they may be made in various shapes (see, for example, FIGS. 13A-C below).

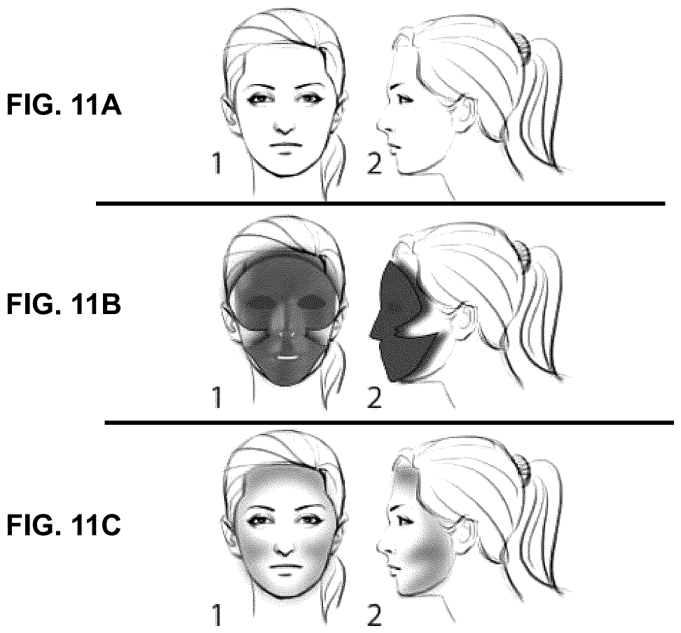

Turning to FIGS. 11A-11C, FIG. 11A illustrates an exemplary front view (1) and exemplary side view (2) of a user's face prior to using a face mask contour system of the disclosure. It should be noted that although FIGS. 11A-11C illustrate the use of the face mask contour system 700, the same effective results can also be achieved for the face mask contour system 100 and the face mask contour system 600. FIG. 11B illustrates the front view (1) and side view (2) of the user's face of FIG. 11A during the use of a face mask contour system of the disclosure. FIG. 11C illustrates the front view (1) and side view (2) of the user's face of FIG. 11A after the use of a face mask contour system of the disclosure, showing the ending results.

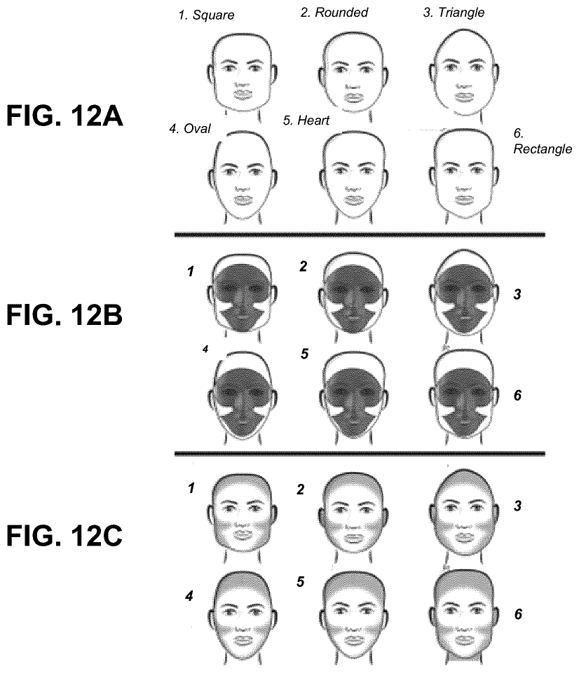

Turning to FIGS. 12A-12C, FIG. 12A illustrates exemplary front views (1-6) of various face and head shapes (e.g., 1. Square, 2. Rounded, 3. Triangle, 4. Oval, 5. Heart, and 6. Rectangle) of various users prior to using a face mask contour system of the disclosure. It should be noted that although FIGS. 11A-11C illustrate the use of the face mask contour system 700, the same effective results can also be achieved for the face mask contour system 100 and the face mask contour system 600. FIG. 12B illustrates the front views (1-6) of the various face and head shapes of FIG. 12A during the use of a face mask contour system of the disclosure. FIG. 12C illustrates the front views (1-6) of the various face and head shapes of FIG. 12A after the use of a face mask contour system of the disclosure, showing the ending results.

Turning to FIGS. 13A-13C, FIG. 13A illustrates exemplary front views (1-5) of various face and head shapes (e.g., 1. Square, 2. Round, 3. Triangle, 4. Diamond, and 5. Heart) of various users prior to using different face mask contour systems of the disclosure. FIG. 13B illustrates the front views (1-5) of the various exemplary face and head shapes of FIG. 13A during the uses of exemplary face mask contour systems of the disclosure. Different users may choose to use different faceplates having different shapes and/or sizes. FIG. 13C illustrates the front views (1-5) of the various face and head shapes of FIG. 13A after the uses of the different exemplary face mask contour systems of the disclosure, showing the ending results.

One or more of the components, processes, features, and/or functions illustrated in the figures may be rearranged and/or combined into a single component, block, feature or function or embodied in several components, steps, or functions. Additional elements, components, processes, and/or functions may also be added without departing from the disclosure. The apparatus, devices, and/or components illustrated in the Figures may be configured to perform one or more of the methods, features, or processes described in the Figures. The algorithms described herein may also be efficiently implemented in software and/or embedded in hardware.

Note that the aspects of the present disclosure may be described herein as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

The enablements described above are considered novel over the prior art and are considered critical to the operation of at least one aspect of the disclosure and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus, if an element can be understood in the context of this specification as including more than one meaning, then its use must be understood as being generic to all possible meanings supported by the specification and by the word or words describing the element.

The definitions of the words or drawing elements described above are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

In the foregoing description and in the figures, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used above, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B”, when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, processes, operations, values, and the like.

It should be noted that where a discrete value or range of values is set forth herein (e.g., 5, 6, 10, 100, etc.), it is noted that the value or range of values may be claimed more broadly than as a discrete number or range of numbers, unless indicated otherwise. Any discrete values mentioned herein are merely provided as examples.

1. A contour mask system for applying contouring solution or tanning, comprising:
a faceplate having a front side and a back side, wherein the faceplate is adapted to cover a first plurality of regions of a face of a user and to leave a second plurality of exposed skin, and wherein the faceplate includes two elongated apertures adapted to fit over the eyes of the user;
an annular ridge encircling each elongated aperture and protruding outwardly from the back side of the faceplate; and
a spacer adapted to create a space gap between the faceplate and the user's face.

2. The contour mask system of claim 1, wherein the spacer includes one or more flexible nose rests fixedly coupled to each elongated aperture.

3. The contour mask system of claim 1, wherein the faceplate has rounded edges.

4. The contour mask system of claim 1 further includes a removable ultraviolet ray protective eyewear.

5. The contour mask system of claim 1, wherein the faceplate is adapted to expose a cheek region, a forehead region and a jawline region of the face of the user, while not exposing a mouth region and a lower cheek region of the face of the user.

6. The contour mask system of claim 1, wherein the faceplate is made of ultraviolet ray protective material.

7. The contour mask system of claim 1, wherein the faceplate is made of vacuum pressed, molded Acrylonitrile Butadiene Styrene plastic.

8. The contour mask system of claim 1, wherein the faceplate is made of machine grade aluminum.

9. The contour mask system of claim 1, wherein the faceplate is made of different sizes.

10. The contour mask system of claim 1, wherein the faceplate is made of different shapes.

11. A method for creating a shadow effect on a face of a user using a contour mask system, wherein the contour mask system comprises a faceplate having a front side and a back side, wherein the faceplate is adapted to cover a first plurality of regions of a face of a user and to leave a second plurality of exposed skin, and wherein the faceplate includes two elongated apertures adapted to fit over the eyes of the user, an annular ridge encircling each elongated aperture and protruding outwardly from the back side of the faceplate, and a spacer, the method comprising:
placing the faceplate to fit over the face of the user;
adjusting the spacer so that when placing the back side of the faceplate over the face of the user, the spacer creates a space gap between the face of the user and the back side of the faceplate; and
applying a sprayed solution over the second plurality of exposed skin of the face of the user.

12. The method of claim 11, wherein the faceplate is adapted to expose a cheek region, a forehead region and a jawline region of the face of the user, while not exposing a mouth region and a lower cheek region of the face of the user.

13. The method of claim 11, wherein the spacer includes one or more flexible nose rests fixedly coupled to each elongated aperture.

14. The method of claim 11, wherein the faceplate has rounded edges.

15. The method of claim 11, wherein the faceplate further includes a removable ultraviolet ray protective eyewear.

16. The method of claim 11, wherein the faceplate is made of ultraviolet ray protective material.

17. The method of claim 11, wherein the faceplate is made of vacuum pressed, molded Acrylonitrile Butadiene Styrene plastic.

18. The method of claim 11, wherein the faceplate is made of machine grade aluminum.

19. The method of claim 11, wherein the faceplate is made of different sizes.

20. The method of claim 11, wherein the faceplate is made of different shapes.
